(19) United States
(12) Patent Application Publication
(10) Pub. No.: US 2024/0005439 A1
(43) Pub. Date: Jan. 4, 2024

(54) IMAGING METHODS AND RADIOTRACERS FOR USE THEREIN

(71) Applicant: VESTLANDETS INNOVASJONSSELSKAP AS (VIS), Bergen (NO)

(72) Inventors: Olav TENSTAD, Bergen (NO); Tom Christian Holm ADAMSEN, Bergen (NO)

(21) Appl. No.: 18/255,557

(22) PCT Filed: Dec. 2, 2021
(86) PCT No.: PCT/EP2021/083936

§ 371 Date: Jun. 1, 2023

(30) Foreign Application Priority Data
Dec. 2, 2020 (GB) 2019006.2

(51) Int. Cl. G06T 1/00; G06T 7/00; A61K 51/08

(52) U.S. Cl. CPC G06T 1/0007; G06T 7/00; A61K 51/088; G06T 2207/10104; G06T 2207/30084

(57) ABSTRACT

A method of imaging a subject, the method comprising the steps: administering a radiotracer to the subject such that the radiotracer enters the bloodstream of the subject; allowing the radiotracer to accumulate in a kidney of the subject; and imaging said kidney using positron emission tomography (PET); wherein the radiotracer comprises a protein labelled with a 6-[18F]fluoropyridin-3-ylcarboxy group. The disclosure also relates to radiotracers and compositions suitable for use in said method, as well as to processes, kits and cassettes for preparing said radiotracers.

### FIELD

The present disclosure relates to methods of renal imaging by positron emission tomography. The disclosure also relates to radiotracers and compositions suitable for use in such methods, as well as to processes, kits and cassettes for preparing said radiotracers.

### BACKGROUND

Glomerular filtration rate (GFR), i.e. the volume of fluid filtered by the renal glomeruli per unit time, is a generally accepted measure of global kidney function. Perturbations of GFR are a hallmark of kidney injury, and the degree of reduction in GFR defines and classifies chronic kidney disease (CKD).

Imaging modalities such as computer tomography (CT), magnetic resonance imaging (MRI) and single photon emission tomography (SPECT) have been used to measure kidney function both in human and experimental animals. An advantage of these techniques is that they enable evaluation of individual kidney function. However, local and regional assessment of GFR using such techniques is not feasible because of limitations regarding temporal and spatial resolution of the detection system and/or because they rely on the use of markers that are excreted in urine.

Of the available imaging methods, positron emission tomography (PET) has sufficient spatial resolution to differentiate between the functional cortex, which contains the filtering glomeruli, and the medulla. Radiotracers that are excreted in urine, such as ¹⁸F-fluorodeoxyglucose (¹⁸F-FSG), ¹⁸F-fluorine and ¹⁸F-fluorodeoxysorbitol (¹⁸F-FSG), have been used to evaluate kidney function by PET in clinical or preclinical settings.

However, the scanning protocol is time-consuming because filtration and excretion of the radiotracers must occur before imaging can take place.

WO 2010/066843 discloses biomolecule complexes which are taught as being useful for evaluating GFR by PET. The use of PET to evaluate GFR has also been reported by Han et al. (Annual Meeting of Scandinavian Physiological Society, 2019).

However, there remains a need for improved methods of renal imaging by PET, as well as improved radiotracers for use in such methods. In particular, there remains a need for methods and radiotracers which provide for an improved signal and/or which permit the rapid assessment of kidney function. There also remains a need for radiotracers which can be used at lower doses, which can be readily synthesized, and/or which are not modified by the labelling procedure.

### SUMMARY

In a first aspect, the present disclosure provides a method of imaging a subject, the method comprising the steps:
administering a radiotracer to the subject such that the radiotracer enters the bloodstream of the subject;
allowing the radiotracer to accumulate in a kidney of the subject; and
imaging said kidney using positron emission tomography (PET);
wherein the radiotracer comprises a protein labelled with a 6-[¹⁸F]fluoropyridin-3-ylcarboxy group.

In a second aspect, the present disclosure provides a radiotracer comprising a protein labelled with a 6-[¹⁸F]fluoropyridin-3-ylcarboxy group, wherein the protein is a ligand for the megalin receptor and/or the cubilin receptor.

In other aspects, the disclosure provides radiotracer compositions comprising the radiotracers described herein, as well as processes, kits and cassettes for preparing the radiotracers. The disclosure also relates to uses of the radiotracers and compositions for PET imaging.

### BRIEF DESCRIPTION OF THE DRAWINGS

FIG. 1 shows: (A) a blood vessel (BV) imaged by PET; (B) a diagram of the cylinder on which the blood vessel is modelled; and (C) the correlation between the area under the curve (AUC) and the sphere volume of interest (VOI) centered on the large abdominal blood vessels.

FIG. 2 shows HPLC chromatograms of unlabelled and ¹⁸F-labelled cytochrome C. Shown are: (A) UV 214 nm detection of unlabelled cytochrome C; and (B) gamma B flow cell detection of purified ¹⁸F-cytochrome C.

FIG. 3 shows size exclusion HPLC data obtained from unlabelled cystatin C (left panel) and ¹²⁴I-labelled cystatin C (right panel). Also shown is the exclusion property of the column (middle panel), where the logarithm of the molecular weights of protein standards are plotted as function of their elution volumes.

FIG. 4 shows the distribution and excretion of ¹⁸F-labelled cytochrome C. Shown are: (A) a graph showing radioactivity over time, where radioactivity is presented as standard uptake volume (SUV); (B) static 2D PET-CT images averaged from 0-30 minutes; and (C) a static 3D PET-CT image averaged from 0-30 minutes.

FIG. 5 presents a comparison of GFR as measured by PET imaging and iohexol clearance. Shown are: (A) GFR determined by PET, calculated as kidney activity divided by area under the curve; (B) GFR calculated based on iohexol injection dose divided by area under the curve; (C) repeated measurements of PET- and iohexol-based GFR for male Wistar rats; and (D) repeated measurements of PET- and iohexol based GFR for SD female rats. Individual values and mean±standard deviation (SD) are shown.

FIG. 6 depicts: (A) GFR over time during disease development, as measured using PET and iohexol clearance; and (B) the plasma creatinine concentration as measured on days 21, 35 and 49. Values are mean±SD.

FIG. 7 presents a comparison of methods for GFR determination in rats. Shown are: (A) correlation between individual values for GFR obtained by PET (PET-GFR) and iohexol (iohexol-GFR) in healthy rats and in rats given adenine to induce CKD (r²=0.96); and (B) Bland Altman analysis showing that the methods have high agreement and that the bias of the method is −0.06, and 95% limitation of the agreement is from −0.53 to 0.40.

FIG. 8 depicts filtration in different cortical zones in rats. The figure shows the zonal intensity in the outer cortex (OC), inner cortex (IC) and corticomedullary area (CM): (A) during control conditions; and (B) after 3 weeks of an adenine diet which resulted in chronic kidney disease. Also shown is: (C) the IC/OC ratio after 3 weeks of the adenine diet.

FIG. 9 depicts GFR in mice as measured by PET. Shown are: (A) a PET-CT image with ¹⁸F-labelled cytochrome C showing an immediate peak in abdominal blood vessels followed by a rapid accumulation in kidney cortex with low extra renal activity; and (B) a comparison of GFR measured by PET and iohexol clearance, from which it can be seen that GFR was not significantly different between the two methods (t-test p=0.5).

### DETAILED DESCRIPTION

The present disclosure provides methods (both diagnostic and non-diagnostic) of renal imaging by PET. The disclosure also provides radiotracers suitable for use in such methods, as well as processes, kits and cassettes for preparing said radiotracers. The methods and radiotracers disclosed herein provide for various advantages. In particular, the radiotracers may exhibit rapid selective kidney uptake and hence may be used to measure GFR in a short period of time (e.g., less than 10 minutes). Further, since the radiotracers are sampled in the proximal tubular cells of their parent glomeruli rather than in urine, local GFR can be accurately measured with a high signal-to-background ratio and without urine sampling. Further, the calculations of GFR using the present method are consistent with those obtained by measuring iohexol clearance, which is a generally accepted reference method. In addition, the present method is minimally invasive and allows for repeated measurements of single kidney GFR and intracortical filtration distribution.

Radiotracers

Disclosed herein are radiotracers comprising a protein labelled with a 6-[¹⁸F]fluoropyridin-3-ylcarboxy group. The use of ¹⁸F as a PET radioisotope provides for a number of advantages. For instance, the radiotracers may have a lower positron energy and a higher positron yield as compared to other radiotracers, which results in better spatial resolution regarding physical imaging characteristics at a lower administered dose. Further, since ¹⁸F has a short half-life (approximately 110 minutes) and can be produced in large quantities by cyclotron production, the radiotracers are particularly suited to use in hospitals and other clinical settings.

The protein is preferably freely filtered by the glomeruli. In an embodiment, the protein has a molecular weight of less than 20 kDa, more preferably less than 15 kDa.

The protein preferably accumulates in the kidney by absorption in the proximal tubular cells of the kidney. In an embodiment, the protein acts as a ligand for the megalin receptor and/or the cubilin receptor. These receptors are multiligand binding receptors found in the plasma membrane of the proximal tubular cells of the kidney.

Megalin can be complexed with cubilin. Ligands for megalin, cubilin and the megalin-cubilin complex include cytochrome C, cystatins, aprotinin, chymotrypsinogen A, lysozyme, ovalbumin and ribonucleases, as well as fragments and variants thereof.

In an embodiment, the protein is selected from cytochrome C, cystatin C, aprotinin, lysozyme, and fragments and variants thereof.

In an embodiment, the protein is selected from cytochrome C, cystatin C and fragments and variants thereof. These proteins are freely filtered by the kidney and are completely and rapidly reabsorbed by the proximal tubules.

In an embodiment, the protein is cystatin C or a fragment or variant thereof. Cystatin C is a cysteine proteinase inhibitor produced by nucleated cells and has a molecular weight of 13.3 kDa. The protein is freely filtered by the glomeruli and then reabsorbed by the proximal tubules, where it is catabolized. Cystatin C is particularly preferred for imaging in humans since it meets the criteria for a GFR marker and is present in all human fluids.

In an embodiment, the protein is cytochrome C or a fragment or variant thereof. Cytochrome C is an oxidoreductase having a molecular weight of 12.4 kDa. Cytochrome C has one or several heme c groups bound to the protein by one, or more commonly two, thioether bonds involving sulfhydryl groups of cysteine residues. The protein is freely filtered at the glomerular membrane, following which it is rapidly taken up by the proximal tubular cells by endocytosis. It has been found that ¹⁸F-labelled cytochrome C appears rapidly in the kidney cortex following intravenous injection, with its concentration increasing rapidly before levelling off as the tracer is removed from plasma. Free tracer may appear in urine after a delay of 10-15 minutes due to lysosomal degradation of filtered ¹⁸F-labelled cytochrome C in the proximal tubular cells. Moreover, it has been found that ¹⁸F-labelled cytochrome C does not suffer from problems of dimer formation or protein binding, which can result in an underestimation of GFR.

In an embodiment, the protein is in substantially monomeric form. In a preferred embodiment, at least 90% (e.g., at least 95% or at least 99%) by weight of the protein is in monomeric form. The extent to which the protein is in monomeric form can be determined using techniques known in the art, such as size exclusion chromatography.

In the radiotracers of the present disclosure, the protein is labelled by a 6-[¹⁸F]fluoropyridin-3-ylcarboxy group. The chemical structure of this group is shown by the formula (I) below:

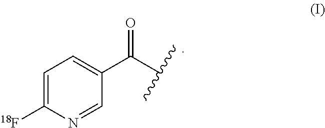 [SMILES: *C(=O)c1ccc([18F])nc1]

In an embodiment, the 6-[¹⁸F]fluoropyridin-3-ylcarboxy group is attached to an amino group of the protein. Accordingly, in this embodiment, the labelled protein may be represented by the following chemical formula (II):

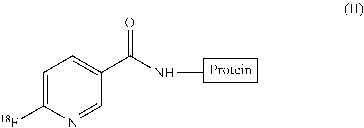

wherein “Protein” denotes the protein and the NH group depicted in said formula denotes an amino group of the protein to which the 6-[¹⁸F]fluoropyridin-3-ylcarboxy group is attached.

In an embodiment, the amino group is an N-terminal amino group or an amino group present on a lysine side-chain. Preferably, the amino group is an N-terminal amino group.

In the radiotracers of the disclosure, a 6-[¹⁸F]fluoropyridin-3-ylcarboxy group may be bound to a single amino acid residue of the protein or to two or more amino acid residues thereof. Preferably, only a single amino acid residue is labelled with said group.

### Processes, Kits and Cassettes for Preparation of the Radiotracers

The radiotracers may be prepared by contacting the protein with an ester of 6-[¹⁸F]fluoronicotinic acid under conditions such that the protein undergoes a reaction with said ester, thereby labelling the protein with a 6-[¹⁸F]fluoropyridin-3-ylcarboxy group.

In particular, the radiotracers may be prepared by contacting the protein with an ester of 6-[¹⁸F]fluoronicotinic acid under conditions such that an amino group of the protein undergoes an acylation reaction with said ester, thereby labelling the protein with a 6-[¹⁸F]fluoropyridin-3-ylcarboxy group.

The labelling reaction may be performed in a solvent such as an aqueous buffer having a pH of from 2 to 11 and at temperature of from 5 to 70° C., preferably at ambient temperature. The labelled protein can then be purified if desired (e.g., by gel and/or ion exchange chromatography).

Preferably, the ester of [¹⁸F]fluoronicotinic acid is 6-[¹⁸F]fluoronicotinic acid 2,3,5,6-tetrafluorophenyl ester, the chemical structure of which is shown by formula (III) below:

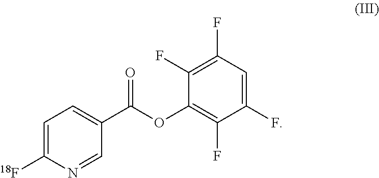 [SMILES: O=C(Oc1c(F)c(F)cc(F)c1F)c1ccc([18F])nc1]

This compound may in turn be prepared by reacting [¹⁸F] fluoride with a compound of the formula (IV):

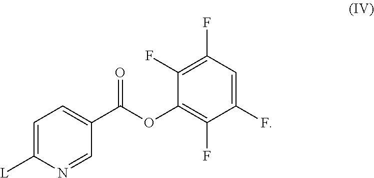

or a salt thereof, wherein L is a suitable leaving group. For instance, L may be selected from chloro, bromo, iodo, nitro, and tri(C₁₋₆alkyl)ammonium. Preferably, L is trimethyl ammonium with a suitable counterion such as a trifluoromethanesulfonate counterion.

This reaction may be performed by standard ¹⁸F-labelling methods. [¹⁸F]fluoride can be conveniently prepared from ¹⁸O-enriched water using the (p,n)-nuclear reaction (see Guillaume et al, Appl. Radiat. Isot. 1991, 42, 749-762) and generally isolated as a salt such as Na¹⁸F, K¹⁸F, Cs¹⁸F, tetraalkylammonium [¹⁸F]fluoride, or tetraalkyl-phosphonium [¹⁸F]fluoride. The reaction may be performed in the presence of a suitable organic solvent such as acetonitrile, dimethylformamide, dimethyl sulfoxide, dimethylacetamide, tetrahydrofuran, dioxane, 1,2-dimethoxyethane, sulpholane, N-methylpyrrolidininone, or in an ionic liquid such as an imidazolium derivative (for example 1-ethyl-3-methylimidazolium hexafluorophosphate), a pyridinium derivative (for example, 1-butyl-4-methylpyridinium tetrafluoroborate), a phosphonium compound, or tetraalkylamonium compound at ambient temperature. To increase the reactivity of the [¹⁸F]fluoride, a phase transfer catalyst such as an aminopolyether or crown ether may be added and the reaction performed in a suitable solvent. These conditions give reactive fluoride ions. Optionally, a free radical trap may be used to improve fluoridation yields. The resulting compound of formula (I) may be purified by standard methods, typically using solid phase extraction, from which the compound can be eluted using an organic solvent/water mixture.

Suitable procedures for preparing the above-mentioned esters and for labelling proteins therewith are disclosed in Olberg et al., J. Med. Chem., 2010, 53, 1732-1740 and in WO 2010/114723.

The disclosure also provides a kit for preparing a radiotracer of the disclosure, the kit comprising:
a first container comprising a solid support on which an ester of nicotinic acid is immobilized, wherein a leaving group is present at the 6-position of the pyridine ring of the nicotinic acid, and wherein the first container is adapted such that a solution comprising [¹⁸F]fluoride can be introduced into the container and reacted with the immobilized ester to form an ester of 6-[¹⁸F]fluoronicotinic acid; and
a second container comprising a protein, wherein the protein is a ligand for the megalin receptor and/or the cubilin receptor.

The immobilized ester present in the first container may be a compound of the formula (IV) shown above. Preferably, the first container is adapted such that the immobilized ester can be contacted directly with a solution comprising [¹⁸F]fluoride produced from a cyclotron so as to form an ester of 6-[¹⁸F]fluoronicotinic acid.

The kit may further comprise a third container comprising a buffer suitable for conducting an acylation reaction between the protein and an ester of [¹⁸F]fluoronicotinic acid and/or instructions for using the kit.

In an embodiment, the process for preparing the radiotracer is an automated process. In this regard, [¹⁸F]-radiotracers may be conveniently prepared in an automated fashion by means of an automated radiosynthesis apparatus. There are several commercially available examples of such apparatus, including FASTlab™ and TRACERlab™ (both from GE Healthcare Ltd.). The apparatus are designed for single-step fluorinations with cyclotron-produced [¹⁸F]-fluoride.

Automated radiosynthesis apparatus commonly comprise a cassette, often disposable, in which the radiochemistry is performed. The cassette is fitted to the apparatus in order to perform the radiosynthesis. The cassettes normally include fluid pathways, a reaction vessel, and ports for receiving reagent vials as well as any solid-phase extraction cartridges used in post-radiosynthetic clean up steps.

The present invention therefore also provides a cassette suitable for use with an automated radiosynthesis apparatus. The cassette comprises:
a vessel containing an ester of nicotinic acid, wherein a leaving group is present at the 6-position of the pyridine ring of the nicotinic acid; and
a vessel containing a protein, wherein the protein is a ligand for the megalin receptor and/or the cubilin receptor.

The cassette may also comprise one or more additional components selected from solid-phase extraction cartridges, filters, reagents, buffers and solvents.

Radiotracer Compositions, Dosing and Administration

The present disclosure also relates to radiotracer compositions comprising a radiotracer of the disclosure and a pharmaceutically acceptable excipient, diluent or carrier.

Radiotracer compositions are typically sterile, pyrogen-free compositions which lack compounds which produce toxic or adverse effects. The compositions preferably comprise a liquid carrier, in which the radiotracer can be suspended or preferably dissolved, such that the composition is physiologically tolerable, i.e. such that it can be administered to the body without toxicity or undue discomfort. The carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g., phosphate buffer); an aqueous solution of one or more tonicity-adjusting substances (e.g., salts of plasma cations with biocompatible counterions), sugars (e.g., glucose or sucrose), sugar alcohols (e.g., sorbitol or mannitol), glycols (e.g., glycerol), or other non-ionic polyol materials (e.g., polyethylene glycols or propylene glycols and the like). Preferably the carrier is pyrogen-free water for injection, isotonic saline or phosphate buffer. The compositions may contain additional optional excipients such as one or more of an antimicrobial preservative, a pH-adjusting agent, a filler, a radioprotectant, a solubiliser and an osmolality adjusting agent. Further examples of compositions and excipients for use therein can be found in standard pharmaceutical texts, e.g., Remington's “The Science and Practice of Pharmacy”, 23rd edition, 2020; and “Handbook of Pharmaceutical Excipients”, 9th edition, 2020.

The radiotracer and carrier may each be supplied in suitable vials or vessels which comprise a sealed container which permits maintenance of sterile integrity and/or radioactive safety, plus optionally an inert headspace gas (e.g., nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle whilst maintaining sterile integrity. Such containers have the additional advantage that the closure can withstand vacuum if desired and they can withstand pressure changes such as reductions in pressure without permitting ingress of external atmospheric gases, such as oxygen or water vapour.

Preferred multiple dose containers comprise a single bulk vial which contains multiple patient doses, whereby single patient doses can thus be withdrawn into clinical grade syringes at various time intervals during the viable lifetime of the preparation to suit the clinical situation. Pre-filled syringes are designed to contain a single (or unit) dose and are therefore preferably a disposable or other syringe suitable for clinical use. The compositions preferably have a dosage of the radiotracer that is suitable for a single patient and are preferably provided in a suitable syringe or container, as described above.

The compositions may be prepared under aseptic manufacture conditions to give the desired sterile, non-pyrogenic product. It is preferred that the key components, especially the associated reagents plus those parts of the apparatus which come into contact with the imaging agent (e.g., vials) are sterile. The components and reagents can be sterilised by methods known in the art, including sterile filtration, terminal sterilisation using, e.g., gamma-irradiation, autoclaving, dry heat or chemical treatment.

The radiotracer compositions may have a radiochemical purity of at least 90%. As used herein, the term “radiochemical purity” refers to the proportion of radioactivity in the composition attributed to the radiotracer. The remaining radioactivity (if any) may come from unreacted or excess ¹⁸F fluoride anions or any other impurity. In some embodiments, the radiotracer compositions have a radiochemical purity of 95% or more, 98% or more, or 99% or more.

It will be appreciated by one of skill in the art that appropriate dosages of the radiotracers, and radiotracer compositions comprising the radiotracers, can vary from subject to subject. The selected dosage level will depend on a variety of factors including, but not limited to, the radioactivity and specific activity of the particular radiotracer employed, the route of administration, the time of administration, the rate of excretion of the radiotracers, the duration of the imaging, the severity of the condition, and the species, sex, age, weight, condition, general health, and prior medical history of the patient. The amount of radiotracer and route of administration will ultimately be at the discretion of the physician, veterinarian, or clinician, although generally the dosage will be selected to achieve local concentrations at the site of action which achieve the desired effect without causing substantial harmful or deleterious side-effects. Administration can be effected in one dose, continuously or intermittently (e.g., in divided doses at appropriate intervals) throughout the course of imaging. Methods of determining the most effective means and dosage of administration are well known to those of skill in the art and will vary with the composition used for imaging, the purpose of the imaging, the target cell(s) being imaged, and the subject being imaged. Single or multiple administrations can be carried out with the dose level and pattern being selected by the treating physician, veterinarian, or clinician.

A suitable dose of the radiotracer for a human subject may be a dose of 100 MBq or more. In some embodiments, the radiotracer is administered to a human subject at a dose of 200 MBq or more, 300 MBq or more, or 400 MBq or more. In one embodiment, the radiotracer is administered to a human subject at a dose of about 500 MBq.

The radiotracer compositions and compounds disclosed herein may be administered to an animal subject or a human subject. Preferably, the subject is a human subject.

The radiotracer compositions are preferably administered intravenously such that the radiotracer directly enters the bloodstream of the subject. Suitable routes for intravenous administration include administration by injection (e.g., a bolus injection), gravity drip or by infusion.

Imaging Methods

The radiotracers and radiotracer compositions described herein are useful for PET imaging, especially renal PET imaging methods. The disclosure therefore also provides methods of renal imaging by PET in which a radiotracer or radiotracer composition described herein is used. Also disclosed herein is the use of the radiotracers and radiotracer compositions in methods of PET imaging. The imaging methods described herein may be diagnostic or non-diagnostic in nature. The methods may involve imaging one or both kidneys of the subject.

PET is a functional imaging modality used in both clinical and laboratory settings that can generate an image revealing a function of a subject's body based on a distribution of a radiotracer throughout at least a portion of the body. To conduct a PET scan, a radiotracer is administered (preferably by injection) to the subject such that the radiotracer enters the bloodstream of the subject. After a period of waiting for the radiotracer to accumulate in the target organ, the subject is placed in a PET imaging scanner and a PET scan is performed. During the scan a record of the concentration of the radiotracer in the target organ is made as the PET radioisotope undergoes positron emission decay. PET imaging can involve the generation of dynamic and/or still images. The images may be two-dimensional or three-dimensional. PET imaging and computed tomography (CT) imaging can be performed together to create a three-dimensional image of the structure of a portion of the subject's body overlaid with a functional image of the same portion of the subject's body. PET may also be used in combination with magnetic resonance imaging (MRI). Suitable methods and apparatus for conducting PET imaging will be apparent to those in the art.

The imaging methods described herein can be used for diagnostic and non-diagnostic applications. For instance, the methods may be used in oncology, surgical planning, radiation therapy and cancer staging. The methods may also be used in research and development, such as in animals for studying human diseases.

In particular, the imaging methods disclosed herein may be used to determine the glomerular filtration rate in a subject. As explained above, the term “glomerular filtration rate” or “GFR” refers to the volume of fluid filtered by the renal glomeruli of a subject per unit time.

GFR may be calculated by the formula GFR=Q/P, where Q is the accumulated radiotracer activity in the renal cortex and P is the time-integrated tracer activity in plasma from the time of administration of the radiotracer to the time of renal scanning.

In order to determine GFR, renal scanning is performed in a time window where the filtered amount of the tracer is quantitatively retained in the proximal tubular cells (i.e., before digestion in the lysosomes is initiated). This time window depends on the radiotracer used and preferably ranges from about 5 to about 30 minutes, more preferably from about 5 to about 10 minutes.

The present disclosure is further illustrated by the following example, which is provided for illustrative purposes only. The example is not to be construed as limiting the scope or content of the disclosure in any way.

### Example

Materials and Methods

Ethical Approval

All animal experiments were conducted in accordance with the regulations of the Norwegian State Commission for Laboratory Animals, harmonized to be in accordance with the European Convention for the Protection of Vertebrate Animals used for Experimental and Other Scientific Purposes and Council of Europe (ETS 123), and with approval from the AAALAC International Accredited Animal Care and Use Program at University of Bergen, approval #FOTS-ID 10508 (rats) and 13922 (mice).

Animal Experiments

A total of 32 rats were used in this study. The rats were male Wistar (n=14, body weight 405 g-460 g, age 12-20 weeks), female Sprague Dawley (SD) (n=6, body weight 305 g-337 g, age 12 weeks) and male SD rats (n=8, weight 432 g-565 g, age 8 weeks). 8 male C3H mice (age 12 weeks, body weight 32-39 g) were also used in the experiments. All animals were anesthetized with 3% sevoflurane mixed with air throughout the scans and were monitored with regard to breathing and temperature (37° C.) during surgery and PET-CT scanning. All animals were sacrificed with an overdose of anesthetic followed by cervical dislocation.

Preparation of ¹⁸F-Cytochrome C

Cytochrome C from bovine heart was labelled using 6-[¹⁸F]fluoronicotinic acid 2,3,5,6-tetrafluorophenyl ester ([¹⁸F]F-Py-TFP) following the approach described by Olberg et al., J. Med. Chem., 2010, 53, 1732-1740. The resulting labelled protein, which comprised a 6-[¹⁸F]fluoropyridine-3-carboxy group, is referred to herein as “18F-cytochrome C”.

The labelled precursor [¹⁸F]F-Py-TFP was prepared and purified by the following procedure. 5 mg of N,N,N-trimethyl-5-((2,3,5,6-tetra-fluorophenoxy)carbonyl)-pyridin-2-aminiumtrifluoro-methanesulfonate was dissolved in 250 μL MeCN (anhydrous, Sigma-Aldrich) and 250 μL t-BuOH (anhydrous, Sigma-Aldrich). 5 μL triethylamine (Sigma-Aldrich) was then added. [¹⁸F]F⁻, which was produced using a cyclotron (GE PETrace 840, Uppsala, Sweden), was trapped on Chromafix PS+(Macherey-Nagel) and dried with 2 mL MeCH (anhydrous, Sigma-Aldrich). The precursor solution was slowly passed through Chromafix/[¹⁸F]F⁻ matrix for radiolabelling in about 3 minutes. The crude product was diluted with 20% acetic acid (VWR) and then passed through a tC18 cartridge (Waters Corp.), previously conditioned with 5 mL MeCN followed by 5 mL water. The tC18 cartridge was then washed with 5 mL 30% MeCN (Sigma-Aldrich) followed by 5 mL water. The product was eluted with 1 mL diethyl ether through a SepPak Dry (Waters Corp.) cartridge and the ether was evaporated. The purified labelled precursor ([¹⁸F]F-Py-TFP) was then dissolved in 30 μL DMSO (Sigma-Aldrich).

Radiolabeling of cytochrome C with [¹⁸F]F-Py-TFP was performed by protein-precursor conjugation in PBS/DMSO at 40° C. for 15 minutes. Unreacted precursor was subsequently removed by size exclusion chromatography (SEC). The purity of the labelled protein was determined through radio-HPLC.

In more detail, 0.4 mg cytochrome C was dissolved in 200 μL of phosphate buffer (50 mM, pH 8-9; Sigma-Aldrich). To this solution, [¹⁸F]F-Py-TFP dissolved in 30 μL DMSO (total 230 μL) was added. The resulting mixture was then placed in a ThermoMixer C (Eppendorf) at 40° C., 300 rpm for 15 minutes. The mixture was then separated on a PD MiniTrap G-10 (GE Healthcare) and 0.5 mL of a 0.9% NaCl solution (Fresenius Kabi) was added. 2-drop fractions were then collected and the activity measured. The fractions on the elution curve on positive gradient were the purified labelled protein.

Preparation of ¹²⁴I-Labelled Cystatin C

Human cystatin C was purchased from Nordic BioSite (Catalog number PPT-20082 Bulk) and labelled with ¹²⁴I for PET by Iodo-Gen as described previously (see Wiig et al., J. Physiol., 2005, 569 (Pt 2), 631-641).

Assessment of Stability of Labelled Proteins

The stability and plasma protein binding of stock solutions of ¹⁸F-cytochrome C and ¹²⁴I-cystatin C was evaluated by high resolution size exclusion chromatography (SEC-HPLC) after 12 h storage in room temperature and after 8 days of storage in fridge (4° C.).

Contrast CT Scanning

Contrast CT images were acquired using a combined small-animal PET CT scanner (nanoScan, Mediso Medical Imaging System, Budapest, Hungary). Wistar males (n=8) were scanned in the supine position, with the center field of view placed over the kidneys. Iohexol (2.5 ml with concentration 350 mg/ml) was injected i.v. in the tail lateral vein with the speed of 1 ml/min by an infusion pump, throughout the scan. Low resolution, semicircular CT images were acquired using an energy of 50 kVp, 300 ms exposure time and 480 projections. Images were reconstructed to an isotropic voxel size of 250 μM, using a RamLak filter. Contrast CT was used to assess the individual imaging kidney cortex volume from the equation: kidney volume=height×length×thickness×π/6. Abdominal blood vessel diameter was also measured by ruler function in contrast CT images. Here the abdominal vessel means the abdominal aorta and vein.

PET GFR Scanning

All animals were scanned using the same scanner, anesthesia procedure, monitoring and positioning as mentioned above. First, CT semicircular scans (same settings as contrast CT) were acquired from the lower edge of the ribs to the pelvis for anatomical reference and attenuation correction for PET. Following CT, a 10-minute dynamic PET acquisition was initiated, which was started 30 seconds before the bolus injection (30 sec duration of the injection) of 5-10 MBq ¹⁸F-cytochrome C diluted in 1 ml saline through a tail vein catheter. Images were reconstructed in 1-5 coincidence mode using the Tera-Tomo 3D reconstruction algorithm (Mediso) with 4 iterations/6 subsets, corrected for attenuation and scatter with a resulting 0.4 mm³ voxel size. The PET acquisition was reconstructed to 15 timeframes: 6×5 s; 6×30 s; 3×120 s.

Ex Vivo Blood Sample During Dynamin PET-CT Scanning

Male Wistar Rats (n=8, weight 448-486 g, age 16 weeks) were equipped with a PE50 catheter in the femoral artery before PET-CT dynamic scanning. During the scanning, seven blood samples were taken at 30 seconds, 1, 2, 4, 6, 7, and 9.5 min. ¹⁸F-cytochrome C activity in plasma was assessed by γ-counting (2480 WIZARD² from PerkinElmer).

Chronic Kidney Disease Animal Model

SD male rats (n=8) were fed by 0.35% adenine in the chow (Altromin, Denmark) for 7 weeks to induce chronic kidney disease (CKD). PET-CT GFR scanning was performed at baseline and at 3, 5 and 7 weeks after initiation of adenine feeding. Iohexol clearance (described below) was performed at baseline and after 3, 5 and 7 weeks of adenine feeding. Blood samples were taken every second week for creatinine measurements.

Measurement of Iohexol Clearance for Rats and Mice

Following isoflurane anesthesia in SD male rats (n=8), the left and right lateral tail vein were catheterized and 300 μl of iohexol (350 mg/ml, Omnipaque, GE Healthcare) was prefilled in a silicon tubing and injected by a peristaltic pump through the left tail vein for 60 seconds and washed with 1000 μl saline containing 20 mg/ml HSA to ensure reproducible injection dose and compensation for blood loss due to the ensuing blood sampling. Blood samples (50 μl) were taken from the right lateral vein catheter at 1, 2, 5, 15 and 30 min after starting the iohexol injection. Following the 30 min blood sample, the rats woke up and were briefly anesthetized with isoflurane for blood sampling at 60, 90, 120 and 180 min. A 240 minutes blood sample was included for the CKD rats on weeks 3, 5 and 7.

Male mice (C3H, n=8) anesthetized with isoflurane were injected i.v. in the tail vein with 100 μl iohexol (diluted 1:10 from stock solution). 15 minutes following the injection, the mice were re-anesthetized with isoflurane and were kept anesthetized until termination of the experiments. Blood samples (each 20 μl) were collected from the retro-orbital space at 15, 30, 45 and 60 minutes.

Iohexol clearance was calculated by a bi-exponential method as: GFR=Dose/AUC, where AUC is the area under the plasma iohexol concentration curve. In addition, the slope of the second mono-exponential phase of the plasma disappearance curve for iohexol (k) was calculated using blood samples taken at 60, 90, 120, and 180 minutes.

PET and CT Imaging Analysis:

All images were analyzed using Inter View Fusion version 3.01.021.0000 (Mediso), and CT and PET images were automatically aligned. The sphere selection volume of interest (VOI) function was used to collect the signal from the large abdominal vessels close to the distal aorta branch level in the frame with the highest intensity and copied to all other frames to obtain the ¹⁸F-cytochrome C intensity as a function of time (input function).

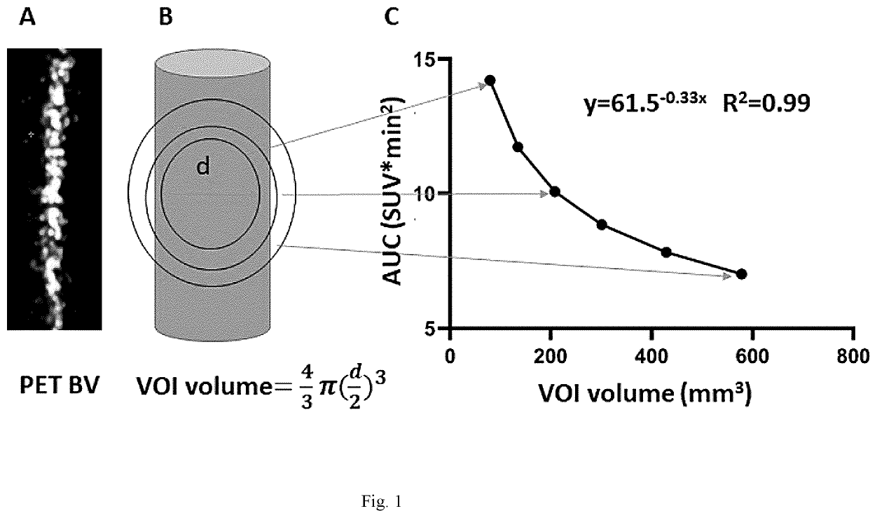

In more detail, 6 input function candidates were generated from 6 sphere-VOIs with diameters of 5, 6, 7, 8, 9, and 10 mm centered on the maximal signal intensity from the vessels and used for regression analyses to obtain an unbiased estimate of the area under the input function (AUC). The AUC (0-5.5 minutes) values obtained from the 6 VOIs were plotted as a function of VOI-volumes and showed an excellent exponential correlation (γ=61.5^{-0.33x}R²=0.99) as shown in FIG. 1. An unbiased AUC was obtained from the regression analysis using a VOI-volume (x in regression equation) corresponding to a blood vessel diameter of 2.41 mm as measured by the ruler function with the software on the contrast CT images (n=8 images from 8 rats, SD=0.02). This approach was verified by ex vivo blood and kidney samples in a separate set of rats (n=8), i.e. the ¹⁸F-clearances obtained by ex vivo sampling were not different from those obtained by the PET images. The same approach was adopted for mice using sphere diameters of 1, 1.5, 2, 2.5, 3, 3.5, and 4 mm and abdominal vessel diameter of 0.9 mm (n=8 mice, SD=0.07). The auto segmentation tool was used to collect the ¹⁸F-signals from both kidneys at 5.5 minutes using thresholds that gave VOI-volumes that matched the kidney volumes as measured by contrast CT. The total kidney ¹⁸F-activity was calculated as the mean VOI-intensity multiplied with the kidney cortex volume (total kidney volume from CT×2/3) and divided by AUC to obtain GFR.

Measurements of Creatinine and Iohexol in Plasma

The plasma creatinine concentration in rats exposed to adenine and thus developing CKD was measured by two-dimensional HPLC or using an enzymatic kit (Enzymatic Rat Creatinine Kit (Crystal Chem catalog 80340).

For measurement of iohexol, all blood samples were centrifuged at 2000 g for collection of plasma. Plasma samples (5 μl from rats and 1 μl from mice) were immediately diluted 7.5-60 times by 0.1% (v/v) trifluoroacetic acid (TFA) in water and their iohexol concentrations were measured by two-dimensional HPLC using the Thermo Scientific™ Dionex™ UltiMate™ 3400 Rapid Separation series hardware, column switching and Chromeleon Chromatography Data System software (7.2.10). A ProSwift™ RP-4H 1 mm (D)×50 mm (L) column was used in the first dimension and an Acclaim™ 300 C18 2.1 mm (D)×10 mm (L) column in the second dimension. Plasma proteins were efficiently removed by the first-dimension column and 0.4 ml eluent (0.1% TFA in water) carrying the iohexol content of the sample was allowed to flow on to the second-dimension column at 0.4 ml/minute. Iohexol concentrated on top of the second-dimension column was eluted as a sharp peak without any interfering contaminants in a 3 minutes 0-30% acetonitrile/0.1% TFA (v/v) gradient and quantified by UV-detector at 247 nm. Both columns were washed separately by 99.9% acetonitrile/0.1% TFA, re-equilibrated in 0.1% TFA in water and ready for the next sample 9 minutes after injection. A dilution series of the iohexol injectate stock solution was used to calibrate the UV signal that was linearly correlated (r²=0.994) with the iohexol concentration in the whole range of the observed concentrations.

Data Analysis

Data were expressed as the mean±standard deviation. ANOVA was used to compare the GFR between different PET-CT scans. Linear regression analyses and Bland Altman analysis were used to compare the iohexol- and PET based GFR calculations. Prism 8 (Graph Pad, USA) was used for statistical analysis.

Results

Free tracer and tracer labeling, in vivo stability and plasma protein binding

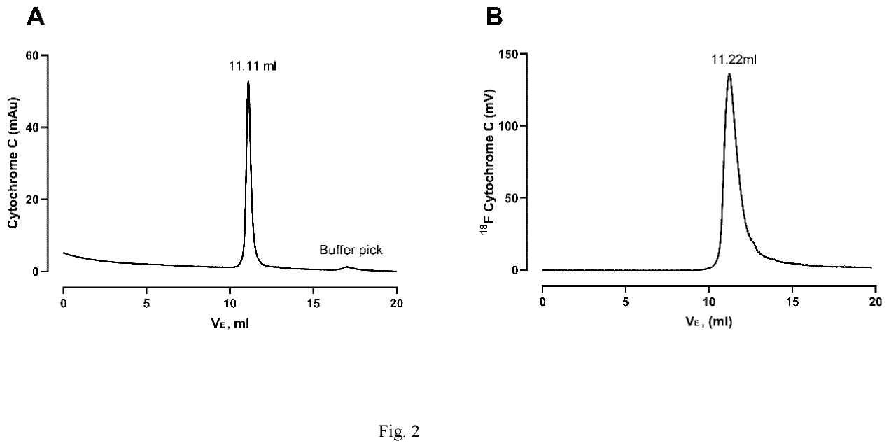

As can be seen from FIG. 2, the elution pattern of ¹⁸F-cytochrome C as evaluated by high resolution size exclusion chromatography was similar to that of unlabelled cytochrome C and there was no sign of dimer formation or protein binding. Further, low molecular weight ¹⁸F-activity in the tracer injected into the animals was less than 1%.

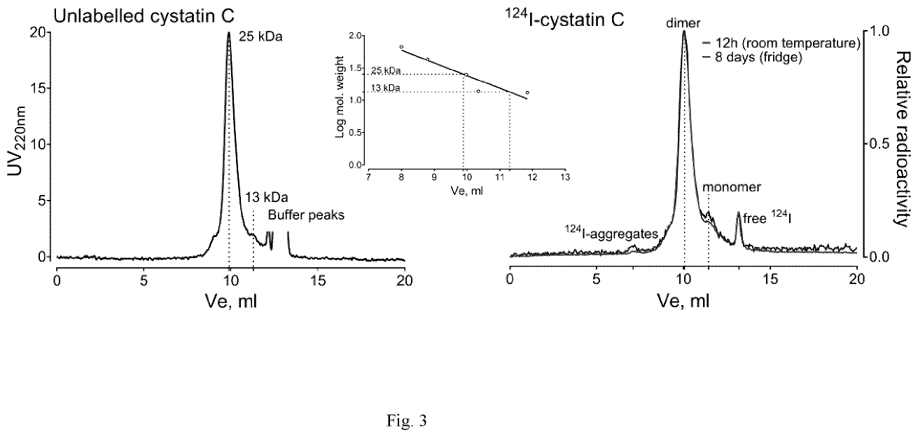

In contrast, it can be seen from FIG. 3 that both unlabelled cystatin C and ¹²⁴I-labelled cystatin C appeared mainly as dimers with an apparent molecular weight of 25 kDa. Only a small fraction appeared in monomeric form (13 kDa). Since dimer formation may result in an underestimation of GFR and the half-life of ¹²⁴I is relatively long (4.2 days), only ¹⁸F-cytochrome C was assessed further in the experiments described herein.

¹⁸F-Cytochrome C Accumulates Selectively in the Kidney During the First 30 Minutes Following Injection

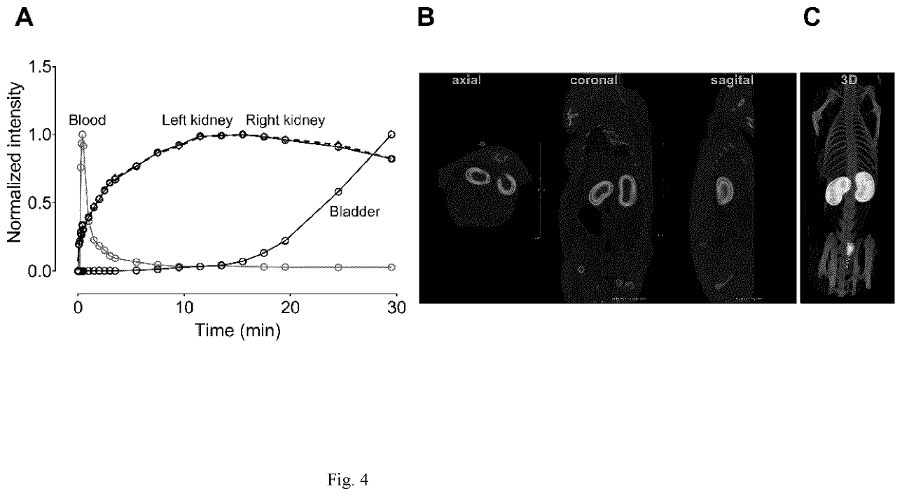

To investigate the initial distribution, uptake and excretion of ¹⁸F-cytochrome C in the body, two rats were dynamically scanned by PET-CT for 30 minutes. As shown in FIG. 4, PET-CT imaging revealed that ¹⁸F-cytochrome C was cleared rapidly from the plasma and exclusively taken up in the kidney following i.v. injection. The ¹⁸F signal peaked over the large abdominal vessels in less than 1 minute and appeared in the bladder in less than 10 minutes. After 10 minutes, the bladder signal increased exponentially and the kidney signal leveled out reflecting urinary excretion of ¹⁸F-cytochrome C breakdown products. These observations suggest that the clearance period should be less than 10 minutes for GFR measurements using ¹⁸F-cytochrome C. Evidently there was an early high renal uptake of ¹⁸F-cytochrome C with negligible involvement of other organs such as liver and spleen suggesting that this tracer is suitable for recording GFR with PET.

Imaging Derived Input Function (IDIF) is Representative for Artery Input Function (AIF)

The arterial input function (AIF) is important for the quantitative analysis of the dynamic PET data. To assess AIF without blood sampling, the abdominal vessel was used as a reference to calculate imaging derived input function (IDIF). This approach was validated by comparing with the AIF. AIF was obtained by taking blood samples and measuring ¹⁸F-cytochrome C during the PET-CT scanning in 8 rats. The ex vivo and image-derived AUCs and renal clearances of ¹⁸F-cytochrome C were compared. There was found to be a linear correlation between the AUCs (r²=0.8) and no differences between the clearances were observed (p=0.8, student's t-test).

No Difference Between PET-CT and Iohexol GFR Measurement in Different Strains, Sexes and Species

To test the feasibility of the PET method and the biological variation in GFR, different strains and sexes of rats and mice were scanned. To test the validity of the method, all the GFR values measured with PET were also compared with iohexol plasma clearance being a generally accepted reference method for GFR determination.

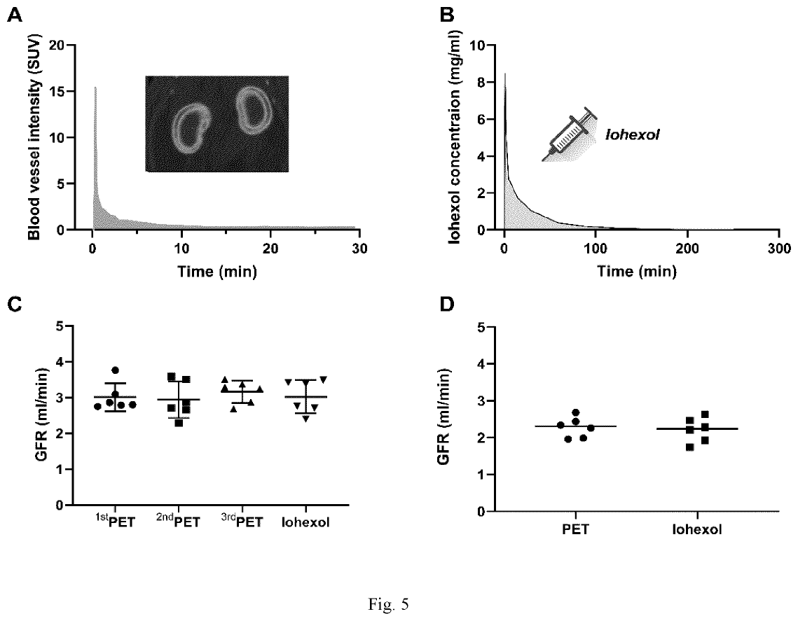

GFR estimations from both PET-CT and iohexol clearances in different strains and sexes are shown in FIG. 5 and Table 1, and demonstrate good reproducibility and correlation between the two methods.

TABLE 1

| | | | | GFR | | | |
|---|---|---|---|---|---|---|---|
| | | Kidney | | | Iohexol/100 g | | GFR/100 g |
| | | volume | | Iohexol | (ml/min · | GFR (PET) | (ml/min · |
| Strains | BW (g) | (mm³) | k (min⁻¹)* | (ml/min) | 100 g) | (ml/min) | 100 g rat) |
| SD female | 310 ± 15 | 856 ± 61 | 0.026 ± 0.002 | 2.25 ± 0.26 | 0.73 ± 0.07 | 1.88 ± 0.16 | 0.61 ± 0.06 |
| (n = 6) | | | | | | | |
| SD male | 491 ± 58 | 1190 ± 71 | 0.025 ± 0.002 | 2.89 ± 0.12 | 0.59 ± 0.06 | 3.20 ± 0.12 | 0.66 ± 0.07 |
| (n = 8) | | | | | | | |
| Wistar | 446 ± 29 | 1412 ± 126 | 0.027 ± 0.002 | 2.84 ± 0.34 | 0.64 ± 0.01 | 3.01 ± 0.35 | 0.68 ± 0.11 |
| male | | | | | | | |
| (n = 6) | | | | | | | |

*Plasma disappearance rate constant of iohexol for the second mono-exponential phase.

As shown in FIG. 5C, there was no difference between the mean GFR calculated by iohexol clearance and 1^{st}, 2^{nd}, 3^{rd} (scanned in three consecutive weeks before iohexol measurement) and PET-CT in Wistar males (n=6) compared by ANOVA. SD females (n=6) were also subjected to PET-CT GFR and iohexol-based GFR, where the same GFR of 2.3±0.3 ml/min was found with both methods (FIG. 5D).

Also, as is evident from Table 1, the plasma k for iohexol was not related to weight or sex. This was also the case for GFR per 100 g body weight as measured by both methods. In the different strains, the PET-CT GFR normalized with the body weight ranged from 0.61 to 0.68 ml/min/100 g (p>0.05, ANOVA).

Similar Decrease in GFR Recorded with PET-CT and Iohexol During Development of CKD

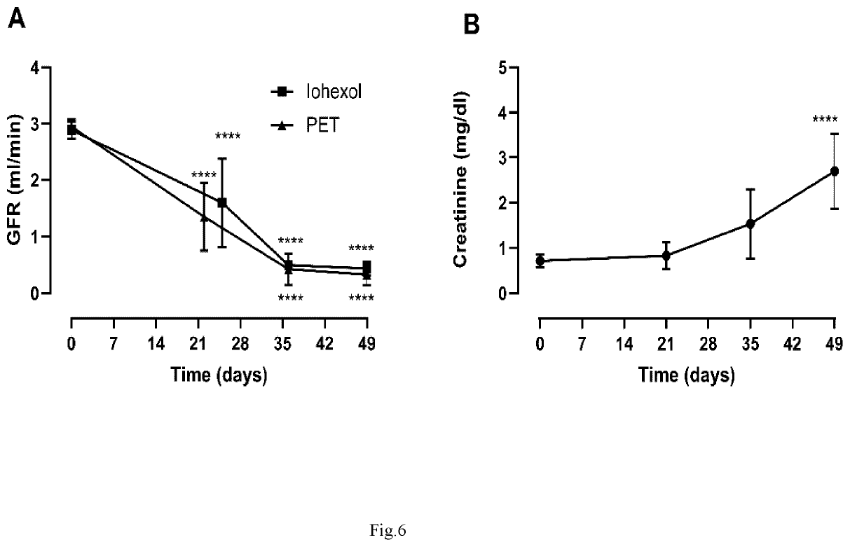

To compare the sensitivity of the PET-CT GFR measurement with the iohexol plasma clearance method in a diseased kidney, GFR was measured in parallel with both methods over time during development of chronic kidney disease induced by 0.35% adenine, an agent known to cause progressive kidney damage and GFR decrease. Mean GFR at baseline estimated from iohexol clearance was 2.89±0.14 ml/min, with a corresponding PET-CT value of 2.94±0.12 ml/min (p>0.05) (FIG. 6). There was a gradual decrease in GFR as recorded with both methods at 3- and 5-weeks following adenine treatment, with values amounting to −50% at 3 weeks and −20% of the initial control value at 7 weeks, respectively (FIG. 6A). The corresponding GFR values measured with the two techniques were not significantly different at any of the time points during disease development (p>0.05).

In addition, kidney function was followed by recording plasma creatinine. The decrease in GFR notwithstanding, the creatinine concentration at 3 weeks was not significantly different from the baseline, but then rose gradually until termination of the experiment at 7 weeks (FIG. 6B). These experiments suggest that ¹⁸F-cytochrome C PET measurement and iohexol clearance estimation have similar sensitivity to diagnose kidney dysfunction.

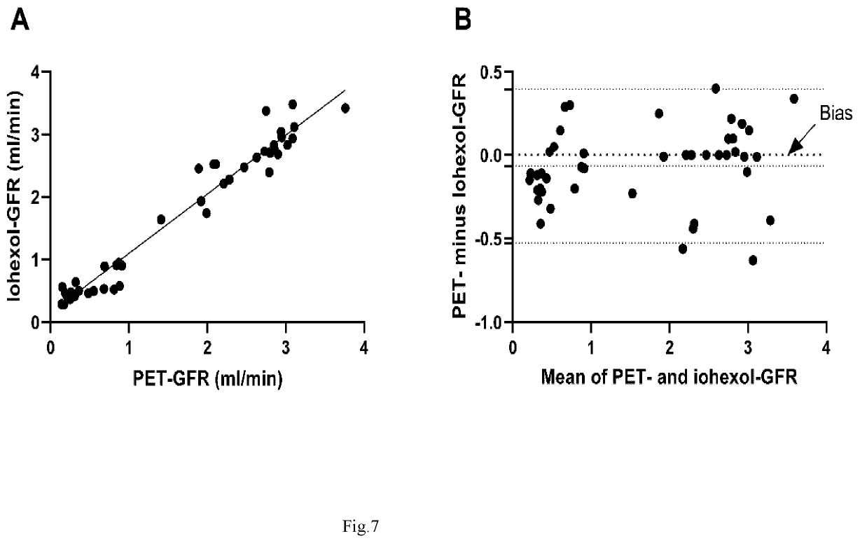

Bland Altman analysis was performed to compare two methods of measurement of GFR. There was good agreement with the two methods in both healthy rats and CKD rats GFR measurement. The bias was −0.06 ml/min (FIG. 7).

Local Filtration on CKD Rats

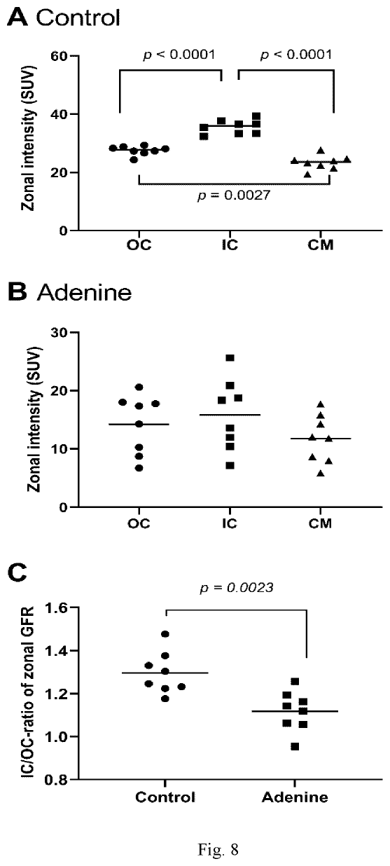

To analyze the local filtration in the rat kidney, small spherical (d=1 mm) VOIs were selected from outer cortex (OC), middle inner cortex (IC) and corticomedullary zone (CM). The OC, IC and CM were defined according to the CT images. Ten-12 VOIs were selected from each layer. As evident from FIG. 8A, there was significantly different filtration in each layer in the control rats. After 3 weeks of the adenine diet, the IC/OC-ratio decreased significantly (FIGS. 8B and 8C) suggesting redistribution of filtration.

Mouse PET-CT GFR

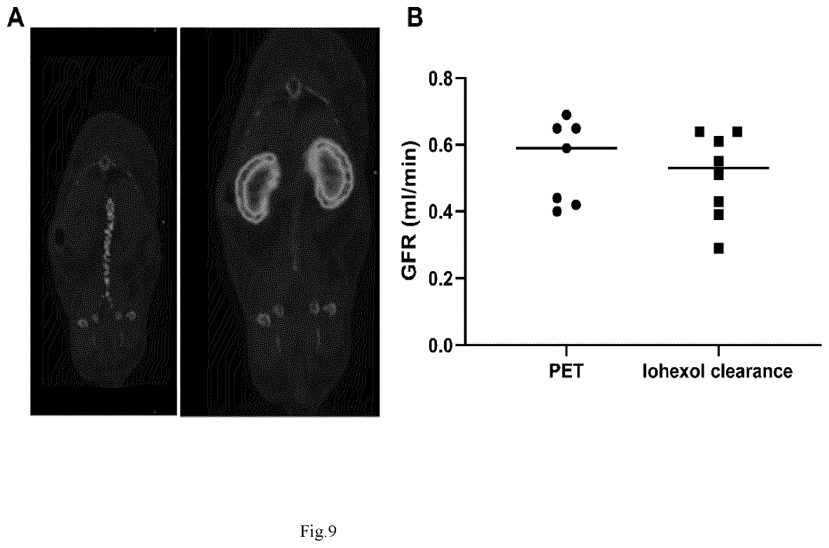

Because of the importance of the mouse as an experimental animal, the PET method was also assessed to determine whether it gave sufficient resolution such that it could be used for GFR assessment in mice. For mice, the input function was determined using the same method as in rats. The blood vessel diameter was measured through CT images, and resulted in an average diameter of 0.9 mm. It turned out that the method was well suited also for use in mice. As shown in FIG. 9, GFR averaged 0.55±0.12 ml/min as measured with PET, which was not different from the GFR of 0.51±0.13 ml/min found with the iohexol plasma clearance method.

Discussion

As this example illustrates, the present method allows for the precise evaluation of local, regional and total GFR within a few minutes and without the need for cumbersome physical blood and urine sampling necessary for conventional urine clearance methods. Results were produced a variety of experimental situations and were indistinguishable from those obtained by the known iohexol clearance method. The calculation of GFR using the present method is straightforward with no need for mathematical modelling, and the reproducibility is good and seemingly within the spontaneous biological variations in GFR over time. The radiotracer is selectively taken up by the kidney and allows for a time window where the cortex content equals the filtered amount before it is degraded and its elements excreted with urine or returned to the circulation. In addition to being minimally invasive, the method also allows for repeated measurements in the same animal in acute short term, as well as in experiments stretching over longer time periods. The method is versatile and, in contrast to the iohexol method, it allows for filtration measurements not only in the single kidney, but also in cortical zones representing functionally different nephrons, i.e. the cortical and juxtamedullary nephrons. The present method therefore allows for an improved assessment of kidney function as compared with existing methods.

The radiotracers described herein also provide for various advantages. For instance, the radiotracers comprise ¹⁸F, which has a short half-life (110 min) and is used clinically such that it can be generated at larger hospitals with PET capabilities. Further, since ¹⁸F is a pure positron emitter, it provides a superior signal with a lower dose as compared to other radioisotopes used in PET. Further, only very small amounts of ¹⁸F are required because the radiotracers accumulate only in filtering nephrons. In a clinical setting, this fact has the obvious benefit that a low radiation dose will be needed. Furthermore, unlike ¹²⁴I-labelled cystatin C, the ¹⁸F-labelled radiotracer used in this study did not suffer from problems of dimer formation or protein binding, which can result in an underestimation of GFR.

All publications, patent applications, patents, and other references mentioned herein are incorporated by reference in their entirety. In addition, the materials, methods, and examples given are illustrative only and not intended to be limiting. Unless otherwise defined, all technical and scientific terms used herein have the same meaning as commonly understood by one of ordinary skill in the art to which this invention belongs. It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the claims.

1. A method of imaging a subject, the method comprising the steps:
administering a radiotracer to the subject such that the radiotracer enters the bloodstream of the subject;
allowing the radiotracer to accumulate in a kidney of the subject; and
imaging said kidney using positron emission tomography (PET);
wherein the radiotracer comprises a protein labelled with a 6-[¹⁸F]fluoropyridin-3-ylcarboxy group.

2. The method of claim 1, wherein the 6-[¹⁸F]fluoropyridin-3-ylcarboxy group is attached to an amino group of the protein, to an N-terminal amino group of the protein, or to an amino group present on a lysine side-chain of the protein.

3. (canceled)

4. The method of claim 2, wherein the protein is labelled by acylating said amino group with an ester of 6-[¹⁸F]fluoronicotinic acid, or with a 6-[¹⁸F]fluoronicotinic acid 2,3,5,6-tetrafluorophenyl ester.

5. (canceled)

6. The method of claim 1, wherein:
(i) the protein is freely filtered by the glomeruli and absorbed by the proximal tubular cells; and/or
(ii) the protein has a molecular weight of less than 20 kDa, e.g., less than 15 kDa; and/or
(iii) the protein is a ligand for the megalin receptor and/or the cubilin receptor; and/or
(iv) wherein the radiotracer comprises said protein in substantially monomeric form.

7-8. (canceled)

9. The method of claim 1, wherein the protein is selected from cytochrome C, cystatin C, aprotinin, lysozyme, and fragments and variants thereof.

10-11. (canceled)

12. The method of claim 1, wherein the radiotracer is administered by intravenous administration.

13. The method of claim 1, wherein the method further comprises determining the glomerular filtration rate (GFR) of the subject based on the acquired PET images.

14. A radiotracer comprising a protein labelled with a 6-[¹⁸F]fluoropyridin-3-ylcarboxy group, wherein the protein is a ligand for the megalin receptor and/or the cubilin receptor.

15. The radiotracer of claim 14, wherein the 6-[¹⁸F]fluoropyridin-3-ylcarboxy group is attached to an amino group of the protein, to an N-terminal amino group of the protein, or to an amino group present on a lysine side-chain of the protein.

16. (canceled)

17. The radiotracer of claim 14, wherein the protein is labelled by acylating said amino group with an ester of 6-[¹⁸F]fluoronicotinic acid, or with a 6-[¹⁸F]fluoronicotinic acid 2,3,5,6-tetrafluorophenyl ester.

18. (canceled)

19. The radiotracer of claim 14, wherein the protein is freely filtered by the glomeruli and absorbed by the proximal tubular cells.

20. The radiotracer of claim 14, wherein the protein is selected from cytochrome C, cystatin C, aprotinin, lysozyme, and fragments and variants thereof.

21. (canceled)

22. The radiotracer of claim 14, wherein the radiotracer comprises said protein in substantially monomeric form.

23. A radiotracer composition comprising a radiotracer of claim 14 and a pharmaceutically acceptable excipient, diluent or carrier.

24. A method of imaging a subject by positron emission tomography (PET) comprising administering a radiotracer of claim 14 to the subject.

25. (canceled)

26. A process for preparing the radiotracer of claim 14, the process comprising:
contacting a protein with an ester of 6-[¹⁸F]fluoronicotinic acid under conditions such that the protein undergoes a reaction with said ester, thereby labelling the protein with a 6-[¹⁸F]fluoropyridin-3-ylcarboxy group;
wherein the protein is a ligand for the megalin receptor and/or the cubilin receptor.

27. (canceled)

28. The process of claim 26, wherein the process is automated.

29. A kit for preparing the radiotracer of claim 14, the kit comprising:
a first container comprising a solid support on which an ester of nicotinic acid is immobilized, wherein a leaving group is present at the 6-position of the pyridine ring of the nicotinic acid, and wherein the first container is adapted such that a solution comprising [¹⁸F]fluoride can be introduced into the container and reacted with the immobilized ester to form an ester of 6-[¹⁸F]fluoronicotinic acid; and
a second container comprising a protein, wherein the protein is a ligand for the megalin receptor and/or the cubilin receptor.

30. A cassette suitable for use with an automated radiosynthesis apparatus, the cassette comprising:
a vessel containing an ester of nicotinic acid, wherein a leaving group is present at the 6-position of the pyridine ring of the nicotinic acid; and
a vessel containing a protein, wherein the protein is a ligand for the megalin receptor and/or the cubilin receptor.
